(19) United States
(12) Patent Application Publication
(10) Pub. No.: US 2022/0047413 A1
(43) Pub. Date: Feb. 17, 2022

(54) BABY SPACE WARMER

(71) Applicant: Emily M. McCurry, Wiggins, CO (US)

(72) Inventor: Emily M. McCurry, Wiggins, CO (US)

(21) Appl. No.: 17/389,082

(22) Filed: Jul. 29, 2021

Related U.S. Application Data
(60) Provisional application No. 63/064,799, filed on Aug. 12, 2020.

(51) Int. Cl. A61F 7/00; H05B 3/28

(52) U.S. Cl. CPC A61F 7/007; H05B 3/286; A61F 2007/0093; A61F 2007/0072; A61F 2007/0088

(57) ABSTRACT

A baby space warmer system includes a baby space warmer having a heating element and/or a power source disposed in a housing and a controller that is operable to control the heating element and/or power source. The controller may control the heating element to warm to a warming temperature, switch to maintaining a maintenance temperature, and then cease heating. The controller may control the heating element in this way using regulation of voltage and/or one or more components, such as one or more timers, thermostats, thermometers, and so on. In some implementations, a baby space warmer system may include a warming container configured to contain the baby space warmer, concentrate heat generated by the baby space warmer to speed heating of the baby space warmer to the warming temperature, retain heat generated by the baby space warmer to maintain the heating temperature and/or the maintenance temperature, and so on.

### CROSS-REFERENCE TO RELATED APPLICATION

This application is a non-provisional patent application of and claims the benefit to U.S. Provisional Patent Application No. 63/064,799, filed Aug. 12, 2020, and titled “Baby Space Warmer,” the disclosure of which is hereby incorporated herein by reference in its entirety.

### FIELD

The described embodiments relate generally to warming devices. More particularly, the present embodiments relate to a baby space warmer.

### BACKGROUND

There is often a great deal of temperature variation in the environment. Even with the use of heating and air conditioning systems and other such temperature control apparatuses, part or all of the environment may not be comfortable for at least some people. For example, a metal bus bench may not retain heat as well as wood or other materials in the winter and people may find such a bench cold upon contact with the body absent sufficient layers of clothing being disposed between.

This may particularly be the case for infants or other small children. Infants or other small children may be more sensitive to temperature variations than adults, and may have less control over, and/or understanding of, their situation. Caregivers for such infants or other small children may need to place the infants or other small children on cold surfaces, such as changing tables, car seats, doctor's scales, and so on, which may be colder enough than the environment to be shocking and/or otherwise uncomfortable to the infants or other small children. This may be upsetting to the infants or other small children, which may be emotionally burdensome on the caregivers and/or make care more challenging.

For example, the warmth of a parent or other caregiver may be a source of comfort for newborn babies. When that parent or other caregiver's warmth is removed for a diaper changing on a cold table, or to weigh the newborn on a scale, to put the newborn on a rocker, and so on, the surface on which the newborn is placed may lack warmth. This may cause the newborn to fuss and/or cry. Even placing a baby in a car seat that has been out in the cold may be unpleasant for the baby as car seats may not be designed to stay warm and/or keep the baby warm.

### SUMMARY

The present disclosure relates to a baby space warmer system. The baby space warmer system may include a baby space warmer having one or more heating elements and/or one or more power sources disposed in a housing and one or more controllers that are operable to control the heating element and/or power source. For example, the housing may be configured as a pad that contains such a heating element. The controller may control the heating element to warm to a warming temperature, switch to maintaining a maintenance temperature, and then cease heating. The controller may control the heating element in this way using regulation of voltage and/or one or more components, such as one or more timers, thermostats, thermometers, and so on. In some implementations, a baby space warmer system may include a warming container. The warming container may be configured to contain the baby space warmer, concentrate heat generated by the baby space warmer to speed heating of the baby space warmer to the warming temperature, retain heat generated by the baby space warmer to maintain the heating temperature and/or the maintenance temperature, provide power to the baby space warmer, provide signals to the baby space warmer to control operation, and/or perform other functions.

In various embodiments, a baby space warming system includes a baby space warmer and a warming container. The baby space warmer includes a housing, a heating element, a battery, and a controller operable to heat the heating element using the battery to a warming temperature, switch to a maintenance temperature, and cease heating the heating element. The warming container is operable to contain the baby space warmer and includes an insulating material operable to concentrate or retain heat generated by the heating element.

In some examples, the heating element includes a resistance wire. In a number of examples, the heating element includes nichrome. In various examples, the insulating material includes at least one of foam or plastic. In some examples, the insulating material includes a vacuum chamber. In a number of examples, the baby space warmer further includes a timer. In various examples, the baby space warmer further includes a power transfer port that is operable to provide power to the battery.

In some embodiments, a baby space warmer includes a housing, a heating element, a battery, and a controller. The controller is operable to heat the heating element using the battery to a warming temperature, switch to a maintenance state, and cease heating the heating element.

In various examples, the baby space warmer further includes a thermometer or thermostat that the controller uses to determine when to switch to the maintenance state or when to cease heating the heating element. In some examples, the baby space warmer further includes a timer that the controller uses to determine when to switch to the maintenance state or when to cease heating the heating element. In a number of examples, the controller is configured to switch to the maintenance state after applying current to the heating element for a time period. In various examples, the controller is configured to cease heating the heating element after applying current to the heating element for a time period. In a number of examples, the housing includes silicone rubber. In various examples, the controller heats the heating element to the warming temperature in less than a minute.

In a number of embodiments, a method of operating a baby space warmer system includes heating a heating element of the baby space warmer to a warming temperature, switching the heating element to a maintenance state, and ceasing heating of the heating element.

In various examples, the method further includes placing the baby space warmer in a warming container. In some examples, the method further includes using the baby space warmer to warm a surface. In a number of examples, the ceasing heating of the heating element is performed upon determining that the heating element exceeds the warming temperature. In various examples, the ceasing heating of the heating element is performed upon determining that a time period has elapsed. In a number of examples, the ceasing heating of the heating element is performed upon switching to the maintenance state.

### BRIEF DESCRIPTION OF THE DRAWINGS

The disclosure will be readily understood by the following detailed description in conjunction with the accompanying drawings, wherein like reference numerals designate like structural elements.

FIG. 1A depicts a caregiver using an example baby space warmer system.

FIG. 1B depicts FIG. 1A after the caregiver opens the warming container of the baby space warmer system.

FIG. 1C depicts FIG. 1B after the caregiver removes the baby space warmer from the warming container.

FIG. 1D depicts FIG. 1C after the caregiver places the baby space warmer on a surface.

FIG. 1E depicts FIG. 1D after the caregiver moves the baby from the carrier to the surface and returns the baby space warmer to the warming container.

FIG. 2A depicts an example baby space warmer.

FIG. 2B depicts the baby space warmer of FIG. 2A after removal of the protective cover.

FIG. 2C depicts the baby space warmer of FIG. 2B with a portion of the housing cut away to reveal internal components.

FIG. 3 depicts a flow chart illustrating a first example method for operating a baby space warmer system. This method may be performed using the baby space warmer system of FIGS. 1A-1E and/or the baby space warmer of FIGS. 2A-2C.

FIG. 4 depicts a flow chart illustrating a second example method for operating a baby space warmer system. This method may be performed using the baby space warmer system of FIGS. 1A-1E and/or the baby space warmer of FIGS. 2A-2C.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The description that follows includes sample systems, methods, and apparatuses that embody various elements of the present disclosure. However, it should be understood that the described disclosure may be practiced in a variety of forms in addition to those described herein.

One solution to the issue of cold surfaces for infants or other small children may be to place blankets and/or other materials on the surfaces. The infants or other small children may then be placed on the blankets. However, such blankets or other materials may cause other issues. To begin with, such blankets or other materials must be transported, kept clean, and so on. Additionally, such blankets or other materials may interfere with the purpose for which the infants or other small children are placed on surfaces. For example, infants or other small children may be placed on the surface of a scale to be weighed and placing such blankets or other materials between the infants or other small children and the surface of the scale may interfere with using the scale to weigh the infants or other small children. Instead, it may be desirable to warm the surface using a system or apparatus that can then be removed before the infants or other small children are placed on the surface.

The following disclosure relates to a baby space warmer system. The baby space warmer system may include a baby space warmer having one or more heating elements and/or one or more power sources disposed in a housing and one or more controllers that are operable to control the heating element and/or power source. For example, the housing may be configured as a pad that contains such a heating element. The controller may control the heating element to warm to a warming temperature, switch to maintaining a maintenance temperature, and then cease heating. The controller may control the heating element in this way using regulation of voltage and/or one or more components, such as one or more timers, thermostats, thermometers, and so on. In some implementations, a baby space warmer system may include a warming container. The warming container may be configured to contain the baby space warmer, concentrate heat generated by the baby space warmer to speed heating of the baby space warmer to the warming temperature, retain heat generated by the baby space warmer to maintain the heating temperature and/or the maintenance temperature, provide power to the baby space warmer, provide signals to the baby space warmer to control operation, and/or perform other functions.

As such, the baby space warmer may not become “hot” as a heating pad becomes hot. The baby space warmer may provide a warmth, such as the temperature to which a wood or metal surface reaches from sustained contact with the human body. The baby space warmer may be placed on surfaces and then removed to warm the surfaces prior to a baby being placed on the warmed surface. This may ease the temperature transition for the baby and reduce the heat/cold difference to provide more comfort for the baby.

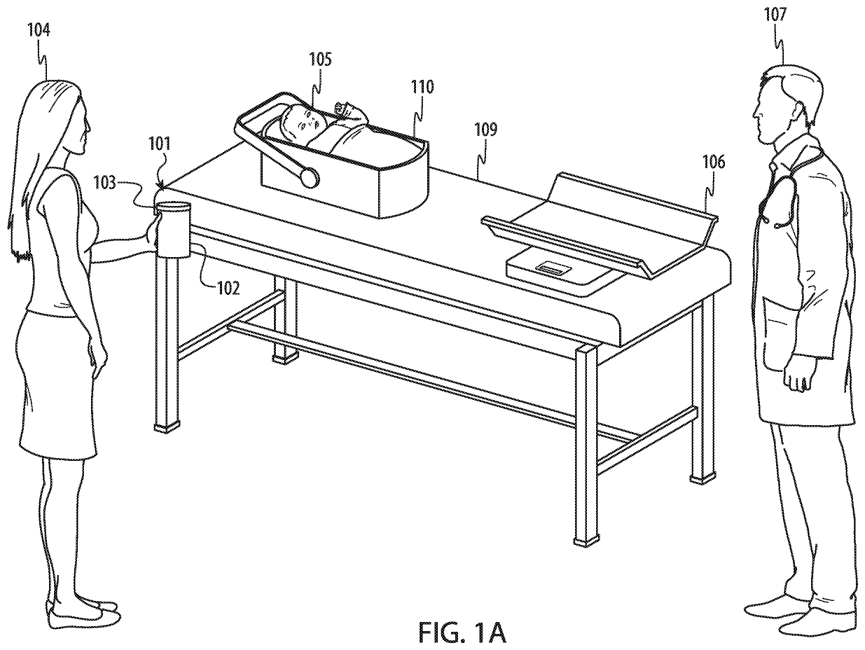

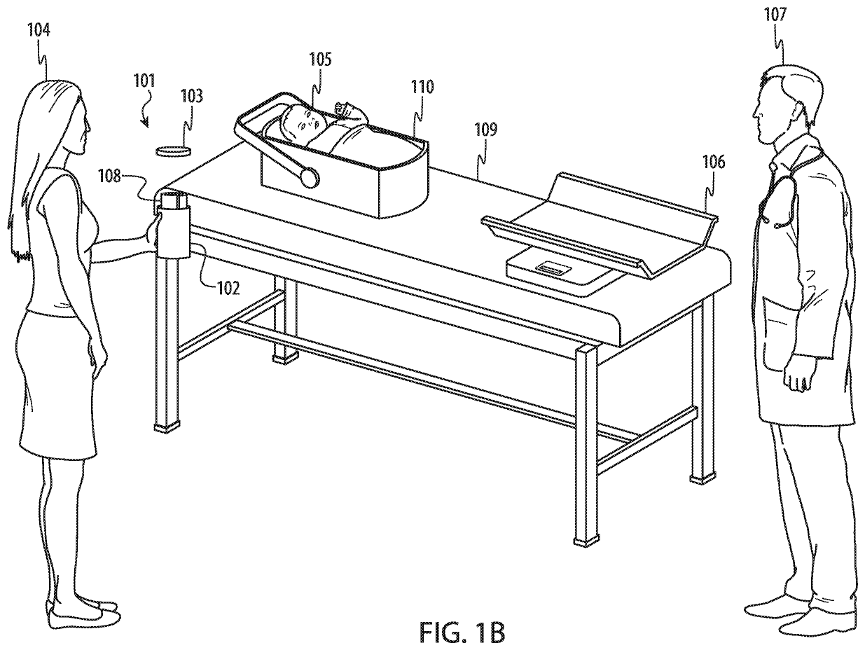

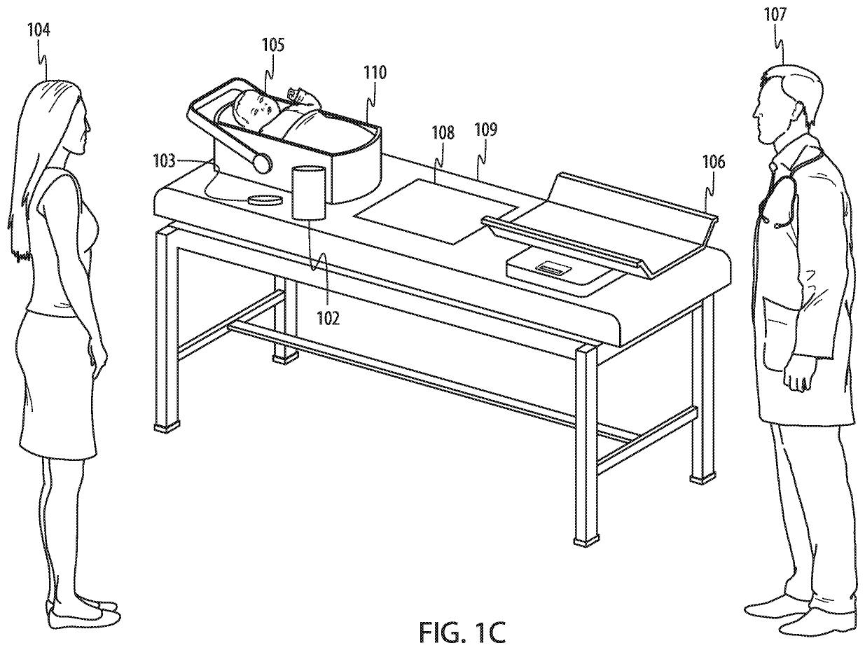

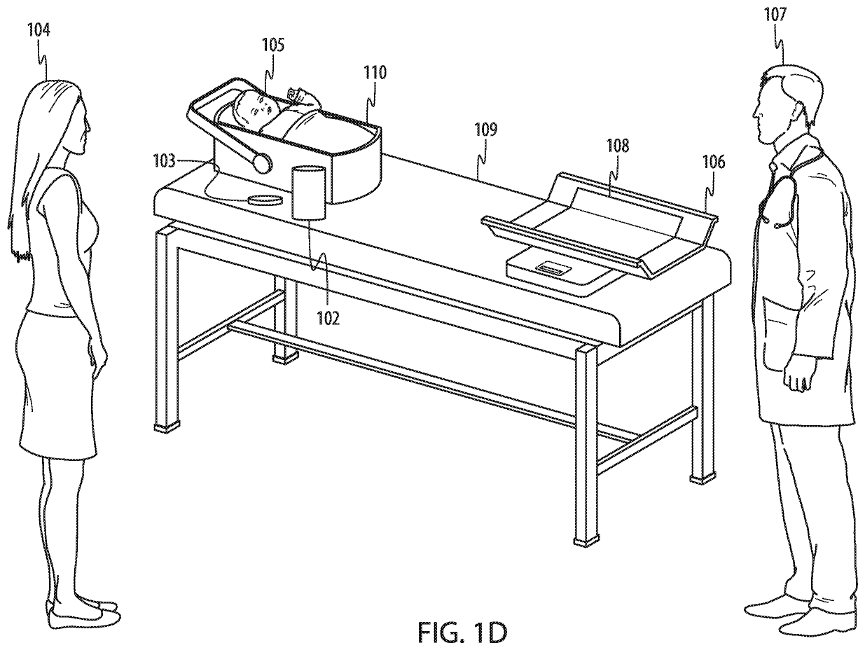

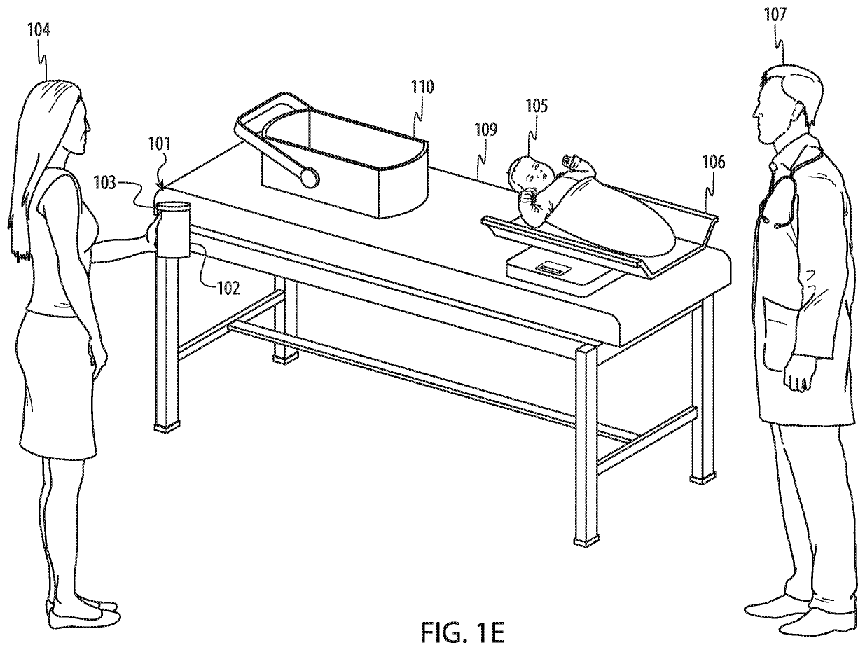

These and other embodiments are discussed below with reference to FIGs. 1A-4. However, those skilled in the art will readily appreciate that the detailed description given herein with respect to these Figures is for explanatory purposes only and should not be construed as limiting.

FIG. 1A depicts a caregiver 104 using an example baby space warmer system 101. As illustrated, the caregiver 104 may have brought a baby 105 in a carrier 110 to the office of a doctor 107. The doctor 107 may be preparing to weigh the baby 105 on a scale 106 atop a table 109. The scale 106 may have a metal, plastic, or other surface that may not retain heat well. As such, the surface of the scale 106 may be “cold” as compared to the ambient environment. The caregiver 104 may use the baby space warmer system 101 to warm the surface of the scale 106 prior to placing the baby 105 on the surface.

In various implementations, the baby space warmer system 101 may include a baby space warmer 108 (see FIGS. 1B-1D) that has one or more heating elements (such as a nichrome wire and/or other conductive element that produces heat via resistance upon application of an electrical current, a chemical heating element, a phase change material heating element, and so on) and/or one or more power sources disposed in a housing and one or more controllers that are operable to control the heating element and/or power source. For example, as shown in FIGS. 1B-1D, the housing may be configured as a pad that contains such a heating element. The controller may control the heating element to warm to a warming temperature (such as between approximately 90 and 100 degrees Fahrenheit), switch to maintaining a maintenance temperature (which may be the same as the warming temperature but may be a maintenance temperature as the controller may keep the heating element at that temperature instead of heating the heating element to that temperature; may be less than the warming temperature, such as 90 degrees Fahrenheit where the warming temperature is 100 degrees Fahrenheit; and so on), and then cease heating. The controller may control the heating element in this way using regulation of voltage and/or one or more components, such as one or more timers, thermostats, thermometers, and so on.

As illustrated, the baby space warmer system 100 may include the warming container 102. The warming container 102 may be configured to contain the baby space warmer 108 (see FIGS. 1B-1D), concentrate heat generated by the baby space warmer 108 to speed heating of the baby space warmer 108 to the warming temperature, retain heat generated by the baby space warmer 108 to maintain the heating temperature and/or the maintenance temperature (which may allow for a previously heated baby space warmer 108 to again reach the warming temperature once turned on again as retaining the heat previously generated by the baby space warmer 108 may necessitate less heating than if the baby space warmer 108 were heated from a fully room temperature and/or otherwise cooler than the retained heat state), provide power to the baby space warmer 108 (such as where the warming container 102 includes a power source and a power transfer component that is operable to provide power to the baby space warmer 108 while the baby space warmer 108 is contained by the warming container 102, such as via a micro USB or other power connector cable that is operable to connect to the baby space warmer 108, an inductive power transmitter that is operable to inductively provide power to an inductive power receiver of the baby space warmer 108, and so on), provide signals to the baby space warmer 108 to control operation (such as where the warming container 102 includes an on or off button that the caregiver 104 can use to signal the baby space warmer 108 to turn on and/or off and/or otherwise control operation of the baby space warmer 108 while the baby space warmer 108 is contained by the warming container 102), and/or perform other functions. For example, the warming container 102 may be formed using one or more insulating materials, such as foam, plastic, a vacuum chamber, another insulating component, and so on. Such insulating materials may concentrate heat generated by the baby space warmer 108 (see FIGS. 1B-1D) so that the baby space warmer 108 reaches the warming temperature faster, retains heat generated by the baby space warmer 108 so that the baby space warmer 108 does not cool as fast as the baby space warmer 108 would otherwise, and so on.

FIG. 1B depicts FIG. 1A after the caregiver 104 opens the warming container 102 of the baby space warmer system 101. In this example, the warming container 102 may include a lid 103 and/or other mechanism operable to contain the baby space warmer 108 in the warming container 102. As shown, such a lid 103 and/or other mechanism may be manipulated to expose the baby space warmer 108 contained by the warming container 102.

The caregiver 104 in this example may have previously activated the baby space warmer 108 to heat to the warming temperature (and/or subsequently maintain the maintenance temperature) such that the baby space warmer 108 is at the warming temperature (and/or the maintenance temperature). The caregiver 104 may now use the baby space warmer 108 to warm the surface of the scale 106 on the table 109 prior to moving the baby 105 from the carrier 110 to the scale 106 so that the doctor 107 can weigh the baby 105.

FIG. 1C depicts FIG. 1B after the caregiver 104 removes the baby space warmer 108 from the warming container 102. As shown, the baby space warmer 108 may be formed as a pad. In some examples, the pad may have a length and width of less than two feet and a thickness of less than ½ inch so as to be portable and easily manipulated. For example, in various examples, the pad may be approximately 16 inches long, 9 inches wide, and approximately ¼ an inch thick. The caregiver 104 may set the warming container 102 and the lid 103 aside on the table 109 so as to be out of the way of the doctor 107 as the warming container 102 and the lid 103 may not be involved in warming the surface of the scale 106.

FIG. 1D depicts FIG. 1C after the caregiver 104 places the baby space warmer 108 on the surface of the scale 106 on the table 109. The baby space warmer 108, having been heated to the warming temperature and/or maintained at the maintenance temperature, may warm the surface of the scale 106. The caregiver 104 may then place the baby space warmer 108 back into the warming container 102, replace the lid 103, move the baby from the carrier 110 to the scale 106, and allow the doctor 107 to weigh the baby 105.

FIG. 1E depicts FIG. 1D after the caregiver 104 moves the baby 105 from the carrier 110 to the surface of the scale 106 on the table 109 and returns the baby space warmer 108 to the warming container 102, closing the lid 103. The doctor 107 may then weigh the baby 105.

Although the baby space warmer system 101 of FIGS. 1A-1E is illustrated and described as including particular components arranged in a particular configuration, it is understood that this is an example. In a number of implementations, various configurations of various components may be used without departing from the scope of the present disclosure.

For example, the baby space warmer system 101 is illustrated and described as including the warming container 102, which includes a lid 103 in this example implementation. However, it is understood that this is an example. In various implementations, otherwise configured warming containers, with or without lids or other components, may be used. In some implementations, the baby space warmer system 101 may omit the warming container 102. Various configurations are possible and contemplated without departing from the scope of the present disclosure.

Additionally, although the baby space warmer system 101 is illustrated and described in the context of warming surfaces for babies, it is understood that this is an example. In some implementations, warming systems may be used to warm surfaces for other purposes. Various configurations are possible and contemplated without departing from the scope of the present disclosure.

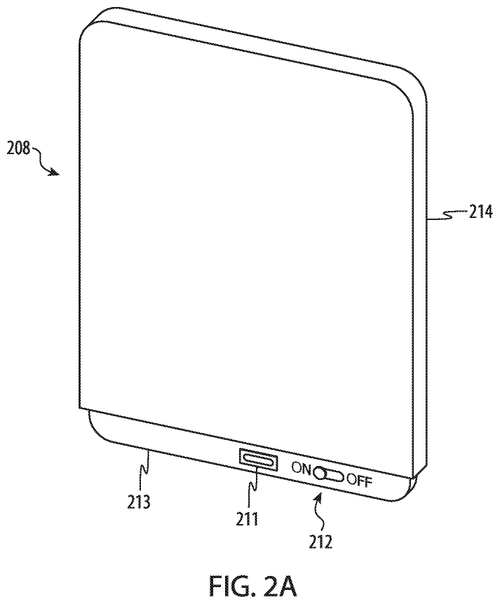

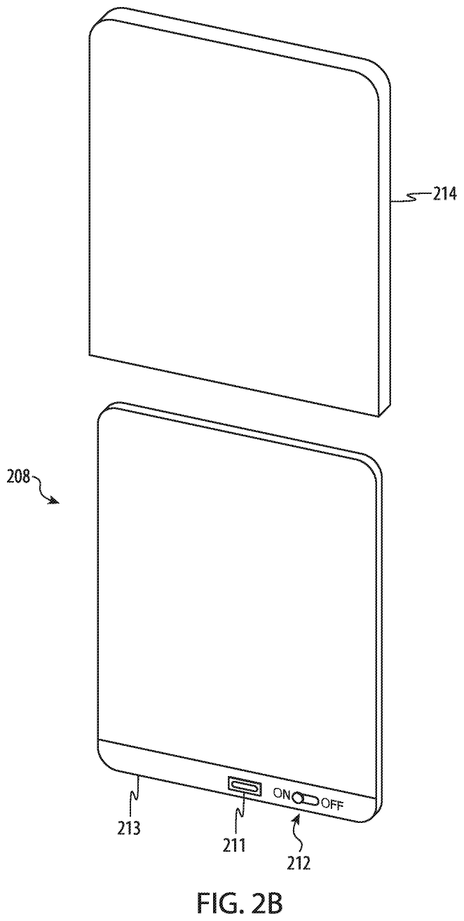

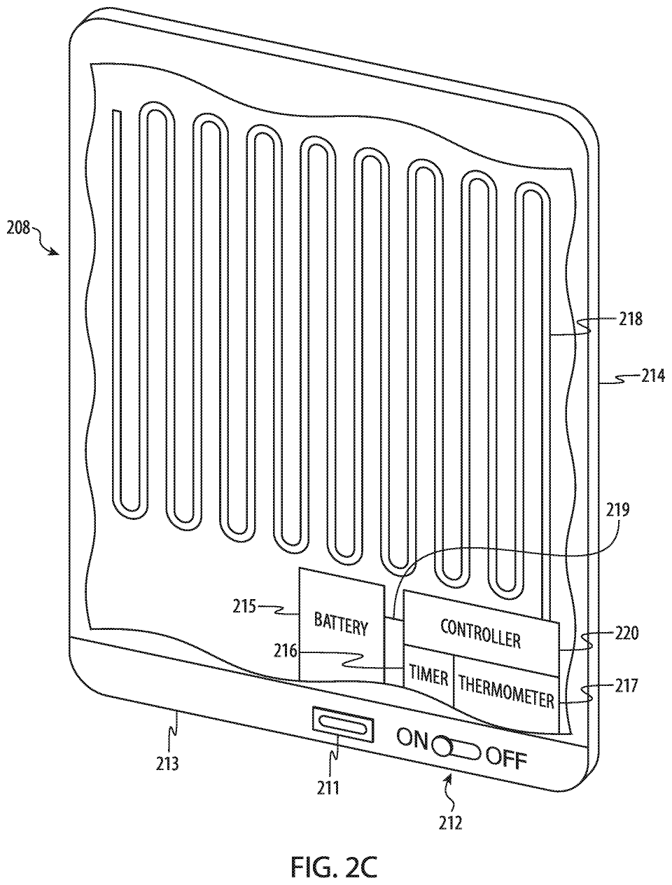

FIG. 2A depicts an example baby space warmer 208. In this example, the baby space warmer 208 may include a housing 213, a removable protective cover 214, a power transfer port 211 (such as a connector port for a micro USB, cellular phone and/or other portable electronic device and/or battery charger, or other power connector cable), and one or more controls 212 (such as an on and/or off switch). The housing 213 may be formed of one or more materials that are operable to contain one or more heating elements and allow heat generated by such heating elements to exit the housing 213. Such materials may be flexible, durable, and easy to clean. Examples of such materials include silicone rubber, plastic, vinyl, and so on. The protective cover may be formed of cloth and/or another material that is operable to protect the baby space warmer 208 from damage or contaminants and may be removed for cleaning and/or replacement.

For example, as shown, the protective cover 214 may be configured to slip on and/or off of the housing 213. FIG. 2B depicts the baby space warmer 208 of FIG. 2A after removal of the protective cover 214. However, it is understood that this is an example. In other examples, the protective cover 214 may include one or more fasteners (such as one or more snaps, buttons, zippers, and so on) that are operable to fasten around the housing 213 such that the protective cover 214 is operable to contain the housing 213 within the protective cover 214.

Although the above illustrates and describes the power transfer port 211 connecting to a micro USB, cellular phone charger, or other power connector cable, it is understood that this is an example. In some implementations, the baby space warmer 208 may instead include an attached power cord and/or power plug. In some examples of such implementations, the baby space warmer 208 may include a compartment that the attached power cord and/or power plug may tuck into for storage when not in use. In other implementations, the baby spaced warmer 208 may include one or more component for receiving power from other kinds of cellular phone and/or other portable electronic device and/or battery charger, such as an inductive power charger receiver. Various configurations are possible and contemplated without departing from the scope of the present disclosure.

FIG. 2C depicts the baby space warmer 208 of FIG. 2B with a portion of the housing 213 cut away to reveal internal components. As shown, the baby space warmer 208 may include a heating element 218. In this example, the heating element 218 may be a resistance wire that is bent into a pattern running across the area enclosed by the housing 213. The resistance wire may be formed of a conductive material, such as nichrome, that produces heat via resistance upon application of an electrical current. However, it is understood that this is an example. In other implementations, other heating elements (such as a chemical heating element, a phase change material heating element, and so on) and/or other configurations of resistance wire may be used without departing from the scope of the present disclosure.

As also shown, the baby space warmer 208 may also include one or more batteries 215 and/or other power sources and one or more controllers 220. The battery 215 may be conductively and/or communicably coupled to the controller 220 via one or more wires and/or other connectors 219. The controller 220 may be operable to apply power from the battery 215 to the heating element 218 and/or otherwise control operation of the baby space warmer 208. For example, the controller 220 may control charging of the battery 215 using power received via the power transfer port 211, detecting an “on” signal from the control 212, detecting an “off” signal from the control 212, heating of the heating element 218 to a warming temperature (such as between approximately 90 and 100 degrees Fahrenheit), switching the heating element 218 to maintain a maintenance temperature (which may be the same as the warming temperature but may be a maintenance temperature as the controller may keep the heating element 218 at that temperature instead of heating the heating element to that temperature; may be less than the warming temperature, such as 90 degrees Fahrenheit where the warming temperature is 100 degrees Fahrenheit; and so on), ceasing the heating element 218 from heating, and so on. The controller 220 may control the heating element 218 in this way using a variety of mechanisms.

For example, the controller 220 may regulate voltage and/or current supplied to the heating element 218. In some implementations, the controller 220 may supply a first amount of current to cause the heating element 218 to quickly heat to the warming temperature, then lessen the current to a second amount of current to maintain the heating element 218 at the maintenance temperature, and then cease applying current to the heating element 218. The controller 220 may be configured with the first and second amounts of current to apply and for how long to cause the heating element 218 to reach the warming temperature after a first desired time, switch to the maintenance temperature after a second desired time, and cease heating after a third desired time without monitoring the actual heat produced by the heating element 218.

Alternatively, as shown, the baby space warmer 208 may include one or more timers 216 and/or thermometers 217 and/or thermostats and/or other components that the controller 220 may use to determine when the warming temperature is reached, determine that the warming temperature is not exceeded, determine when to switch to the maintenance temperature, determine when to cease heating, verify that heating has ceased, verify that the baby space warmer 208 is not too hot, determine when any component may be operating improperly, otherwise control operation of the baby space warmer 208, and so on.

The operation of the baby space warmer 208 may be different than that of a heating pad. Heating pads typically heat to a temperature of approximately 120 degrees Fahrenheit and sustain that heating for a period of approximately twenty minutes or more for the purpose of applying sustained heat to muscle tissue. Due to such temperatures and durations, heating pads typically heat slowly to their operating temperature. However, the baby space warmer 208 may operate for a much smaller amount of time as the baby space warmer 208 may be used to warm a surface instead of applying sustained heat to muscle tissue. Also, the baby space warmer 208 may warm to much lower temperatures than heating pads, such as 100 degrees Fahrenheit as opposed to 120 degrees Fahrenheit. Further, as the baby space warmer 208 may quickly heat to the warming temperature (such as within 30 seconds to a minute) and then switch to a maintenance temperature and maintain that maintenance temperature for a shorter time than heating pads typically operate (such as approximately 7 minutes), the baby space warmer 208 may be significantly less likely to cause fires, burns, and/or other problems than heating pads typically do. As such, it may be safe for baby space warmers 208 to heat to their warming temperatures much faster than it would be safe for a heating pad to heat to the heating pad's operating temperature. This may prevent a caregiver from having to stand around waiting for the baby space warmer 208 to heat to the warming temperature while a baby is awaiting care. However, it is understood that these are examples. Various configurations are possible and contemplated without departing from the scope of the present disclosure.

In various examples, the baby space warmer 208 may include one or more additional and/or different components than those shown. Such components may include one or more processing units and/or other processors, non-transitory storage media (which may take the form of, but is not limited to, a magnetic storage medium; optical storage medium; magneto-optical storage medium; read only memory; random access memory; erasable programmable memory; flash memory; and so on), communication units, inductive power receivers, and/or other components.

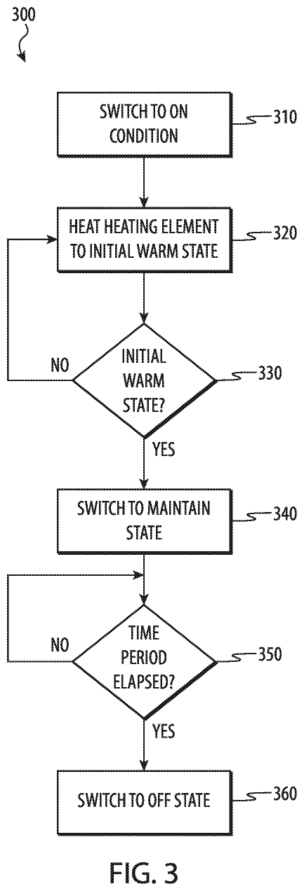

FIG. 3 depicts a flow chart illustrating a first example method 300 for operating a baby space warmer system. This method 300 may be performed using the baby space warmer system 101 of FIGS. 1A-1E and/or the baby space warmer 208 of FIGS. 2A-2C.

At operation 310, a baby space warmer may switch to an “on” condition. The flow may proceed to operation 320 where the baby space warmer may heat a heating element to an initial warm state. The initial warm state may be where the heating element reaches a warming temperature, such as between approximately 90 and 100 degrees Fahrenheit. In some examples, the heating element may be heated to the initial warm state in approximately less than a minute.

At operation 330, the baby space warmer may determine whether or not the initial warm state is reached. If so, the flow may proceed to operation 340. Otherwise, the flow may return to operation 320 and heating of the heating element may continue. In some implementations, a thermometer or thermostat may be used to determine whether or not the initial warm state is reached. In other examples, determination of whether or not the initial warm state is reached may be based upon a length of time that a particular amount of current or voltage is applied to the heating element. Various configurations are possible and contemplated without departing from the scope of the present disclosure.

At operation 340, after the initial warm state is reached, the baby space warmer may switch to a maintain state. The maintain state may be where a maintenance temperature is maintained. In some implementations, the maintenance temperature may be the same as the warming temperature but may be a maintenance temperature as the baby space warmer may keep the heating element at that temperature instead of heating the heating element to that temperature. In other implementations, the maintenance temperature may be less than the warming temperature, such as 90 degrees Fahrenheit where the warming temperature is 100 degrees Fahrenheit. The baby space warmer may apply less current or voltage to the heating element in the maintain state than the initial warm state as less current or voltage may be required for the heating element to maintain the maintain state than is required for the heating element to reach the initial warm state.

At operation 350, the baby space warmer may determine whether or not a time period has elapsed. If not, the flow may return to operation 350 where the baby space warmer may again determine whether or not the time period has elapsed. Otherwise, the flow may proceed to operation 360 where the baby space warmer may switch to an “off” state. As such, the time period may function as a safety in case a caregiver leaves the baby space warmer in operation. For example, the time period may be 8 minutes. In some implementations, the baby space warmer may determine whether or not the time period has elapsed using one or more timers and/or other time determining mechanisms, such as a controller that is configured to apply a current or a voltage to the heating element to maintain the heating element in the maintain state for the time period.

Although the example method 300 is illustrated and described as including particular operations performed in a particular order, it is understood that this is an example. In various implementations, various orders of the same, similar, and/or different operations may be performed without departing from the scope of the present disclosure.

For example, the method 300 is illustrated and described as determining whether or not the initial warm state is reached. However, it is understood that this is an example. In some implementations, the baby space warmer may heat the heating element to the initial warm state without any determination of whether or not that initial warm state is reached. Various configurations are possible and contemplated without departing from the scope of the present disclosure.

By way of another example, the method 300 is illustrated and described as determining whether or not the time period has elapsed. However, it is understood that this is an example. In some implementations, the baby space warmer may switch from the maintain state to the off state without determining whether or not any kind of time period has elapsed. Various configurations are possible and contemplated without departing from the scope of the present disclosure.

In yet another example, the method 300 is illustrated and described as including an on state, an initial warm state, a maintain state, and an off state. However, it is understood that this is an example. In some implementations, the baby space warmer may heat to the warm state when turned on and then immediately switch to the off state. In such an implementation, the maintain state may be where the previously heated baby space warmer cools while in the off state. Various configurations are possible and contemplated without departing from the scope of the present disclosure.

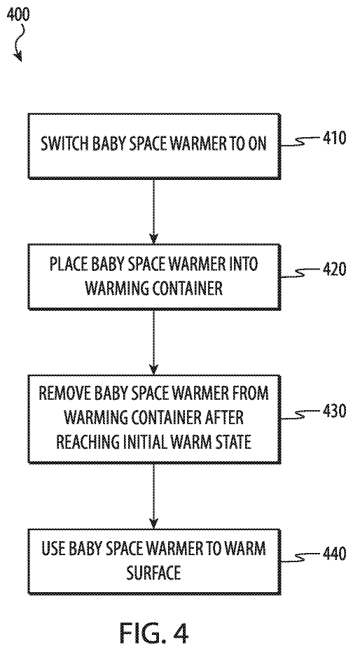

FIG. 4 depicts a flow chart illustrating a second example method 400 for operating a baby space warmer system. This method 400 may be performed using the baby space warmer system 101 of FIGS. 1A-1E and/or the baby space warmer 208 of FIGS. 2A-2C.

At operation 410, a baby space warmer may switched to on. At operation 420, the baby space warmer may be placed into a warming container. At operation 430, the baby space warmer may be removed from the warming container after the baby space warmer reaches the initial warm state. At operation 440, the baby space warmer may be used to warm one or more surfaces.

Although the example method 400 is illustrated and described as including particular operations performed in a particular order, it is understood that this is an example. In various implementations, various orders of the same, similar, and/or different operations may be performed without departing from the scope of the present disclosure.

For example, in some examples, the baby space warmer may be placed back in the warming container after being used to warm the surface. Various configurations are possible and contemplated without departing from the scope of the present disclosure.

By way of another example, the method 400 illustrates and describes placing the baby space warmer in the warming container after the baby space warmer is switched to on. However, it is understood that this is an example. In some implementations, the baby space warmer may be switched to on while contained by the warming container. Various configurations are possible and contemplated without departing from the scope of the present disclosure.

By way of yet another example, the method 400 illustrates and describes removing the baby space warmer from the warming container after the baby space warmer reaches the initial warm state. However, it is understood that this is an example. In some implementations, the baby space warmer may be removed from the warming container prior to reaching the initial warm state. Various configurations are possible and contemplated without departing from the scope of the present disclosure.

In various implementations, a baby space warming system may include a baby space warmer and a warming container. The baby space warmer may include a housing, a heating element, a battery, and a controller operable to heat the heating element using the battery to a warming temperature, switch to a maintenance temperature, and cease heating the heating element. The warming container may be operable to contain the baby space warmer and may include an insulating material operable to concentrate or retain heat generated by the heating element.

In some examples, the heating element may include a resistance wire. In a number of examples, the heating element may include nichrome. In various examples, the insulating material may include at least one of foam or plastic. In some examples, the insulating material may include a vacuum chamber. In a number of examples, the baby space warmer may further include a timer. In various examples, the baby space warmer may further include a power transfer port that is operable to provide power to the battery.

In some embodiments, a baby space warmer may include a housing, a heating element, a battery, and a controller. The controller may be operable to heat the heating element using the battery to a warming temperature, switch to a maintenance state, and cease heating the heating element.

In various examples, the baby space warmer may further include a thermometer or thermostat that the controller uses to determine when to switch to the maintenance state or when to cease heating the heating element. In some examples the baby space warmer may further include a timer that the controller uses to determine when to switch to the maintenance state or when to cease heating the heating element. In a number of examples, the controller may be configured to switch to the maintenance state after applying current to the heating element for a time period. In various examples, the controller may be configured to cease heating the heating element after applying current to the heating element for a time period. In a number of examples, the housing may include silicone rubber. In various examples, the controller may heat the heating element to the warming temperature in less than a minute.

In a number of embodiments, a method of operating a baby space warmer system may include heating a heating element of the baby space warmer to a warming temperature, switching the heating element to a maintenance state, and ceasing heating of the heating element.

In various examples, the method may further include placing the baby space warmer in a warming container. In some examples, the method may further include using the baby space warmer to warm a surface. In a number of examples, the ceasing heating of the heating element may be performed upon determining that the heating element exceeds the warming temperature. In various examples, the ceasing heating of the heating element may be performed upon determining that a time period has elapsed. In a number of examples, the ceasing heating of the heating element may be performed upon switching to the maintenance state.

Although the above illustrates and describes a number of embodiments, it is understood that these are examples. In various implementations, various techniques of individual embodiments may be combined without departing from the scope of the present disclosure.

As described above and illustrated in the accompanying figures, the present disclosure relates to a baby space warmer system. The baby space warmer system may include a baby space warmer having one or more heating elements and/or one or more power sources disposed in a housing and one or more controllers that are operable to control the heating element and/or power source. For example, the housing may be configured as a pad that contains such a heating element. The controller may control the heating element to warm to a warming temperature, switch to maintaining a maintenance temperature, and then cease heating. The controller may control the heating element in this way using regulation of voltage and/or one or more components, such as one or more timers, thermostats, thermometers, and so on. In some implementations, a baby space warmer system may include a warming container. The warming container may be configured to contain the baby space warmer, concentrate heat generated by the baby space warmer to speed heating of the baby space warmer to the warming temperature, retain heat generated by the baby space warmer to maintain the heating temperature and/or the maintenance temperature, provide power to the baby space warmer, provide signals to the baby space warmer to control operation, and/or perform other functions.

In the present disclosure, the methods disclosed may be implemented as sets of instructions or software readable by a device. Further, it is understood that the specific order or hierarchy of steps in the methods disclosed are examples of sample approaches. In other embodiments, the specific order or hierarchy of steps in the method can be rearranged while remaining within the disclosed subject matter. The accompanying method claims present elements of the various steps in a sample order, and are not necessarily meant to be limited to the specific order or hierarchy presented.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A baby space warming system, comprising:
a baby space warmer, comprising:
a housing;
a heating element;
a battery; and
a controller operable to heat the heating element using the battery to a warming temperature, switch to a maintenance temperature, and cease heating the heating element; and
a warming container operable to contain the baby space warmer and comprising an insulating material operable to concentrate or retain heat generated by the heating element.

2. The baby space warming system of claim 1, wherein the heating element comprises a resistance wire.

3. The baby space warming system of claim 1, wherein the heating element comprises nichrome.

4. The baby space warming system of claim 1, wherein the insulating material comprises at least one of foam or plastic.

5. The baby space warming system of claim 1, wherein the insulating material comprises a vacuum chamber.

6. The baby space warming system of claim 1, wherein the baby space warmer further comprises a timer.

7. The baby space warming system of claim 1, wherein the baby space warmer further comprises a power transfer port that is operable to provide power to the battery.

8. A baby space warmer, comprising:
a housing;
a heating element;
a battery; and
a controller operable to heat the heating element using the battery to a warming temperature, switch to a maintenance state, and cease heating the heating element.

9. The baby space warmer of claim 8, further comprising a thermometer or a thermostat that the controller uses to determine when to switch to the maintenance state or when to cease heating the heating element.

10. The baby space warmer of claim 8, further comprising a timer that the controller uses to determine when to switch to the maintenance state or when to cease heating the heating element.

11. The baby space warmer of claim 8, wherein the controller is configured to switch to the maintenance state after applying current to the heating element for a time period.

12. The baby space warmer of claim 8, wherein the controller is configured to cease heating the heating element after applying current to the heating element for a time period.

13. The baby space warmer of claim 8, wherein the housing comprises silicone rubber.

14. The baby space warmer of claim 8, wherein the controller heats the heating element to the warming temperature in less than a minute.

15. A method of operating a baby space warmer system, comprising:
heating a heating element of a baby space warmer to a warming temperature;
switching the heating element to a maintenance state; and
ceasing heating of the heating element.

16. The method of claim 15, further comprising placing the baby space warmer in a warming container.

17. The method of claim 15, further comprising using the baby space warmer to warm a surface.

18. The method of claim 15, wherein the ceasing heating of the heating element is performed upon determining that the heating element exceeds the warming temperature.

19. The method of claim 15, wherein the ceasing heating of the heating element is performed upon determining that a time period has elapsed.

20. The method of claim 15, wherein the ceasing heating of the heating element is performed upon switching to the maintenance state.
